(19) United States
(12) Patent Application Publication
(10) Pub. No.: US 2008/0171087 A1
(43) Pub. Date: Jul. 17, 2008

(54) METHODS AND MATERIALS FOR INCREASING THE ADHESION OF ELUTION CONTROL MATRICES TO SUBSTRATES

(76) Inventors: Ralph A. Chappa, Prior Lake, MN (US); Michael J. Finley, St. Louis Park, MN (US)

Correspondence Address: PAULY, DEVRIES SMITH & DEFFNER, L.L.C., Plaza VII-Suite 3000, 45 South Seventh Street, MINNEAPOLIS, MN, 55402-1630, US

(21) Appl. No.: 11/839,905

(22) Filed: Aug. 16, 2007

Related U.S. Application Data
(60) Provisional application No. 60/822,605, filed on Aug. 16, 2006.

(51) Int. Cl. A61K 9/00; A61L 33/06

(52) U.S. Cl. 424/486; 427/2.1

(57) ABSTRACT

The present invention relates to methods and components for increasing adhesion of an elution control matrix to a polymeric substrate, and medical devices including such components. In an embodiment, the invention includes a medical device including a substrate having a surface, the substrate comprising a polysiloxane, a parylene layer contacting the surface of the substrate, and an elution control matrix contacting the parylene layer, the elution control matrix comprising a polymeric matrix and an active agent dispersed within the polymeric matrix. Other embodiments are included herein.

This application claims the benefit of U.S. Provisional Application No. 60/822,605, filed Aug. 16, 2006, the content of which is herein incorporated by reference.

### FIELD OF THE INVENTION

The present invention relates to methods and materials for increasing the adhesion of coatings to substrates. More specifically, the present invention relates to methods and materials for increasing the adhesion of elution control matrices to polymeric substrates, and medical devices incorporating the same.

### BACKGROUND OF THE INVENTION

Elution control matrices or coatings are now commonly used on medical devices because of the various advantages they can provide. For example, elution control matrices can be configured to control the elution rate of an active agent. As another example, elution control matrices can be disposed on medical devices that can be positioned as desired within the body of a patient. Therefore, elution control matrices can allow active agent delivery to be site-specific, offering therapeutic advantages.

Polymers are frequently used as a substrate for medical devices. Common polymeric substrates for medical devices include polysiloxanes (commonly known as “silicone” or “silicone rubber”). Silicone has beneficial properties such as being flexible, relatively inexpensive, and substantially biocompatible. As a result, many different medical devices are commonly made from silicone including catheters, drainage tubing, introducer tips, flexible sheaths, etc.

Accordingly, there is a need for methods and components that can be used to deposit elution control matrices on polymer substrates such as silicone.

### SUMMARY OF THE INVENTION

Embodiments of the invention include methods and components for increasing adhesion of an elution control matrix to a polymeric substrate, and medical devices including the same. In an embodiment, the invention includes a medical device including a substrate having a surface, the substrate including a polysiloxane, a parylene layer contacting the surface of the substrate, and an elution control matrix contacting the parylene layer, the elution control matrix including a polymeric matrix and an active agent dispersed within the polymeric matrix.

In an embodiment, the invention includes a medical device including a substrate having a surface, the substrate including a polymer, a parylene layer contacting the surface of the substrate, the parylene layer having a thickness of between about 0.01 microns to about 1.0 micron, and an elution control matrix contacting the parylene layer, the elution control matrix including a polymeric matrix and an active agent dispersed within the polymeric matrix.

In an embodiment, the invention includes a method of bonding an elution control matrix to a substrate surface including depositing a parylene layer on the substrate surface, the substrate including a polysiloxane, and depositing an elution control matrix on the parylene layer, the elution control matrix including an active agent.

In an embodiment, the invention includes a medical device including a substrate, the substrate including a polysiloxane, a silane compound bonded to the substrate, and a polymer layer bonded to the silane compound through the residue of one or more latent reactive groups.

The above summary of the present invention is not intended to describe each discussed embodiment of the present invention. This is the purpose of the figures and the detailed description that follows.

### BRIEF DESCRIPTION OF THE FIGURES

The invention may be more completely understood in connection with the following drawings, in which:

FIG. 1 is a cross-sectional view of an elution control matrix disposed on a substrate in accordance with an embodiment of the invention.

FIG. 2 is a cross-sectional view of an elution control matrix disposed on a substrate in accordance with another embodiment of the invention.

FIG. 3 is a cross-sectional view of an elution control matrix disposed on a substrate in accordance with another embodiment of the invention.

FIG. 4 is a cross-sectional view of an elution control matrix disposed on a substrate in accordance with another embodiment of the invention.

FIG. 5 shows a schematic view of a stent in accordance with an embodiment of the invention.

FIG. 6 shows a schematic view of a catheter in accordance with an embodiment of the invention.

While the invention is susceptible to various modifications and alternative forms, specifics thereof have been shown by way of example and drawings, and will be described in detail. It should be understood, however, that the invention is not limited to the particular embodiments described. On the contrary, the intention is to cover modifications, equivalents, and alternatives falling within the spirit and scope of the invention.

### DETAILED DESCRIPTION OF THE INVENTION

The adhesion of an elution control matrix to a substrate may be challenged in vivo in various ways. By way of example, some medical devices are inserted into the body and then later removed. The process of inserting and removing the device can result in substantial mechanical stresses. Similarly, a medical device may be subject to repetitive motion within the body, such as in the case of a medical device located in or near the heart, also producing substantial mechanical stresses.

Failure of adhesion can lead to pieces of the elution control matrix peeling and detaching from the medical device substrate, which can potentially result in adverse consequences. Detachment of a piece of elution control matrix from an intraocular implant could potentially lead to distortion of a patient's vision. Detachment of a piece of elution control matrix from an arterial stent could act as an embolus. For these reasons, the adhesion of an elution control matrix to a substrate is important under many clinical scenarios.

Various polymeric elution control matrices are routinely adhered to metals such as stainless steel and Nitinol, which are common medical device substrate materials. Frequently, adhesion of polymeric coatings directly to metals is sufficiently strong to prevent issues such as peeling of the polymeric coating.

However, it has been discovered that it is difficult to sufficiently adhere elution control matrices to other medical device substrates such as polysiloxanes. Polysiloxanes, or silicones, are commonly used medical device substrate materials because of beneficial properties including being inexpensive, flexible, biocompatible and chemically resistant. However, as shown in examples 1 and 2 below, adhesion of elution control matrices directly to a polysiloxane may not be sufficient to prevent peeling.

It is believed that observed differences in the adhesion of elution control matrices to silicone versus metal substrates are explained in part by the differing surface properties of these substrate materials. Silicone used in medical devices is generally more strongly hydrophobic than stainless steel having a water contact angle of greater than 100 degrees. In contrast, a common medical device metal such as stainless steel (316L) has a water contact angle of between about 45 to 50 degrees. As such, medical device substrates made of silicone are, in general, significantly more hydrophobic than stainless steel. In addition, the chemical groups on the surface of silicones that may impact adhesion through covalent or non-covalent interactions are substantially different than those on the surface of metals.

Further complicating effective adhesion of elution control matrices to silicone, the physical deformation of silicone in response to applied forces is significantly different than the physical deformation of metals such as stainless steel. Specifically, silicones generally deform more readily then most metals. The increased deformation of silicone is believed to lead to increased mechanical forces being exerted on the interface between the elution control matrix and the silicone substrate as the silicone flexes and deforms.

However, as demonstrated in the examples below, a layer of parylene can be used to increase the adhesion of an elution control matrix to a polymeric substrate. Specifically, a layer of parylene can be used to adhere an elution control matrix to a polysiloxane substrate. When a layer of parylene is used to bond the elution control matrix to a polysiloxane substrate, the elution control matrix exhibits greater adhesion to the substrate and enhanced resistance to peeling and flaking. This effect is surprising because polysiloxanes have surfaces that are generally not conducive to adhesion.

Accordingly, embodiments of the invention include methods and components for increasing the adhesion of an elution control matrix to a polymeric substrate, and medical devices including such components. In an embodiment, the invention includes an active agent eluting medical device including a substrate comprising a polysiloxane polymer, a layer of parylene disposed on the surface of the substrate, and an elution control matrix disposed on the parylene layer, the elution control matrix configured to control the elution rate of the active agent. In an embodiment, the invention includes a method for adhering an active agent elution control matrix to a polymeric substrate including depositing a parylene layer over a polymeric substrate and depositing an elution control matrix over the parylene layer.

In addition, as demonstrated in the examples below, linking compounds including one or more latent reactive groups can be used to increase the adhesion of various coatings to a polymeric substrate. The latent reactive groups are capable of generating an active specie, such as a free radical, in response to external stimulation to bond the linking compound to the substrate and/or to the elution control matrix through the residues of the latent reactive groups.

Accordingly, embodiments of the invention can include a medical device including a substrate, a linking compound bonded to the surface of the substrate through the residue of a latent reactive group, and an elution control matrix disposed on the linking compound, the elution control matrix comprising a polymeric matrix, the elution control matrix configured to control the elution rate of the active agent.

Referring now to FIG. 1, a cross-sectional view of a medical device surface 100 is shown in accordance with an embodiment of the invention. A primer layer 104 is disposed on a substrate 102. The substrate 102 can include a polymer. By way of example, the substrate 102 can include a polysiloxane. Exemplary substrate polymers are described in greater detail below.

The primer layer 104 can comprise parylene. The term “parylene” as used herein shall refer to a polymer belonging to the group of polymers based on p-xylylene (substituted or unsubstituted). Parylenes have the repeating structure -(p-CH₂—C₆H₄—CH₂)ₙ—. Common parylene polymers include poly(2-chloro-paraxylylene) (“parylene C”), poly(paraxylylene) (“parylene N”), and poly(2,5-dichloro-paraxylylene) (“parylene D”). In a particular embodiment, the polymer deposited by the method includes poly(2-chloro-paraxylylene) (“parylene C”). parylenes of the invention can also include mono-, di-, tri-, and tetra-halo substituted polyparaxylylenes. In an embodiment, the polymer includes mono-, di-, tri-, and tetra-chloro substituted polyparaxylylene. In an embodiment, the polymer includes mono-, di-, tri-, and tetra-fluoro substituted polyparaxylylene. Other parylene derivatives can include poly(dimethoxy-p-xylylene), poly(sulfo-p-xylylene), poly(iodo-p-xylylene), poly(trifluoro-p-xylylene), poly(difluoro-p-xylylene), and poly(fluoro-p-xylylene).

If the primer layer 104 is too thick, it can stiffen the underlying substrate. This can be undesirable in some applications. This is particularly significant in the context of silicone, such as where silicone is selected as a substrate because of its flexibility. In some embodiments, the primer layer 104 is less than about 10 microns thick. In some embodiments, the primer layer 104 is less than about 1 micron thick. If the primer layer 104 is too thin, it may be difficult to assure uniform coverage of the substrate. In some embodiments, the primer layer 104 is greater than about 0.01 microns thick. In an embodiment, the primer layer 104 is between about 0.01 and about 0.5 microns thick. In a particular embodiment, the primer layer 104 is about 0.1 microns thick.

An elution control matrix 106 can be disposed on the primer layer 104. The term “elution control matrix” as used herein shall refer to a polymeric matrix that is configured to elute an active agent. The elution control matrix 106 can include one or more degradable polymers, one or more non-degradable polymers, or combinations of both. In some embodiments, the elution control matrix 106 can include one or more hydrophobic polymers. Exemplary degradable, non-degradable, and hydrophobic polymers are described in more detail below. The elution control matrix 106 can also include one or more active agents. The elution control matrix 106 can be configured to control the rate at which the active agent is eluted there from. As used herein, the term “active agent” means a compound that has a particular desired activity. For example, an active agent can be a therapeutic compound that exerts a specific activity on a subject. Exemplary active agents can include peptides, proteins, carbohydrates, nucleic acids, lipids, polysaccharides, synthetic inorganic or organic molecules, or combinations thereof that cause a desired biological effect when administered to an animal, including but not limited to birds and mammals, including humans.

The thickness of the elution control matrix 106 can depend on many factors including, for example, the specific polymers used in the matrix, the desired loading of active agent within the elution control matrix 106, the type of medical device being coated, etc. In some embodiments, the elution control matrix 106 is from about 0.5 microns to about 200 microns thick.

Embodiments of the invention can also include methods of adhering an active agent elution control matrix to a polymeric substrate. For example, an embodiment can include depositing a parylene layer over a polymeric substrate and depositing an elution control matrix over the parylene layer.

Deposition of the parylene layer can be performed using various specific techniques. In an embodiment, the parylene layer can be deposited using a vacuum deposition system. In some vacuum deposition systems a polymer quantity is vaporized in a vaporization chamber and then passes through a cracking chamber where parylene dimer vapor is cracked into activated monomer vapor. Vaporized activated monomer is then deposited onto a substrate in a deposition chamber. An exemplary vacuum deposition system is the PDS-2010 LABCOTER® available from Specialty Coating Systems (Indianapolis, Ind.).

The elution control matrix can be deposited onto the parylene layer using any of a variety of coating techniques including dip-coating, spray-coating (including both gas-atomization and ultrasonic atomization), fogging, brush coating, press coating, blade coating, and the like. The coating solution may be applied under conditions where atmospheric characteristics such as relative humidity, temperature, gaseous composition, and the like are controlled. In some embodiments, the coating solution is applied using a spray technique. Exemplary spray coating equipment that can be used to apply matrices of the invention can be found in U.S. Pat. No. 6,562,136; U.S. application Ser. No. 10/409,434; U.S. application Ser. No. 10/256,349; U.S. application Ser. No. 10/976,348; U.S. application Ser. No. 10/976,193; U.S. application Ser. No. 11/102,465; and U.S. App. Ser. No. 60/736,995, the contents of which are all hereby incorporated by reference.

In some embodiments, the primer layer 104 can include a silane compound. Various types of exemplary silane compounds are described in greater detail below. In some embodiments, the elution control matrix 106 can be formed with a polymer having a latent reactive group. As such, the elution control matrix 106 can be bonded to the primer layer 104 through the residue of a latent reactive group.

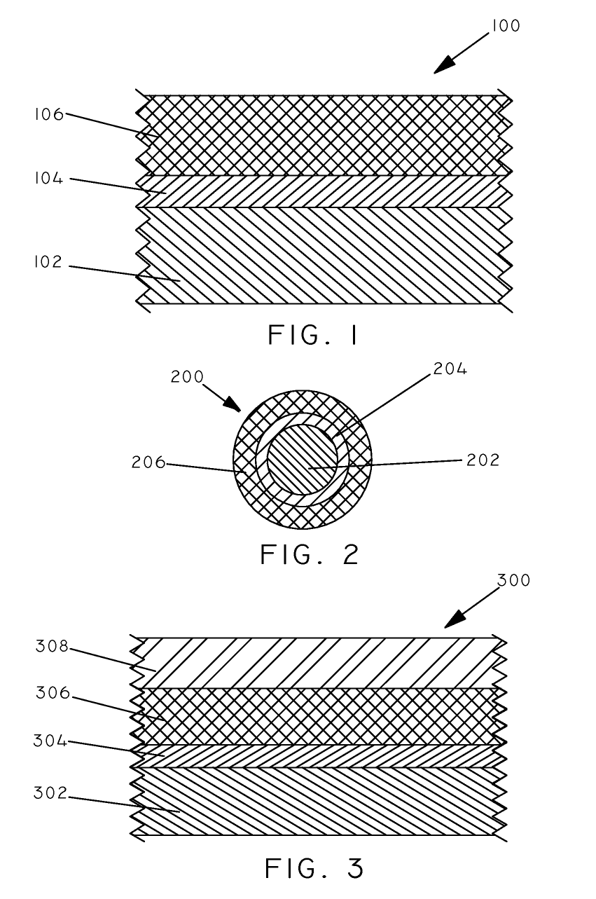

Referring now to FIG. 2, a cross-sectional view of a medical device 200 is shown in accordance with another embodiment of the invention. A primer layer 204 is disposed on a substrate 202. In this example, the medical device substrate 202 is circular in cross-section, such as with a wire or tube, and the primer layer 204 surrounds the medical device surface on all sides. An elution control matrix 206 is disposed on the primer layer 204. The elution control matrix 206 surrounds the primer layer 204 on all sides.

In some embodiments, multiple elution control matrices can be disposed on the primer layer. Referring now to FIG. 3, a cross-sectional view of a medical device 300 is shown in accordance with another embodiment of the invention. A primer layer 304 is disposed on a substrate 302. A first elution control matrix 306 is disposed on the primer layer 304. An active agent can be dispersed within the first elution control matrix 306. A second elution control matrix 308 is disposed on the first elution control matrix 306. In some embodiments, an active agent is also dispersed within the second elution control matrix 308. The active agent in the first elution control matrix 306 can be the same or different than the active agent in the second elution control matrix 308. The first elution control matrix 306 and the second elution control matrix 308 can both include a polymeric matrix. The polymeric matrix can include one or more degradable and/or non-degradable polymers. The polymer(s) of the first elution control matrix 306 can be the same or different than the polymer(s) of the second elution control matrix 308.

Referring now to FIG. 4, a cross-sectional view of a medical device surface 400 is shown in accordance with another embodiment of the invention. A linking layer 404 is disposed on a substrate 402. The linking layer 404 can be formed by bonding a linking compound to the substrate 402. The linking compound can include one or more latent reactive groups. The term “latent reactive group” as used herein shall refer to groups which respond to specific applied external stimuli, such as ultraviolet light, to undergo active specie generation. Latent reactive groups generate active species such as free radicals, nitrenes, carbenes, and excited states of ketones upon absorption of external electromagnetic or thermal energy. Latent reactive groups can include photoreactive groups. The term “photoreactive group” shall refer to those latent reactive groups that are responsive to the ultraviolet and visible portions of the electromagnetic spectrum. Examples of linking compounds and latent reactive groups are described in U.S. Pat. Nos. 5,002,582; 5,414,075; 5,512,329; 5,637,460; 5,714,360; 6,077,698; 6,278,018; 6,603,040; and 6,924,390 the contents of which directed to compounds that can serve as linking compounds and latent reactive groups is herein incorporated by reference.

Latent reactive groups can include azides (such as arylazides, acyl azides, azido formates, sulfonyl azides, phosphoryl azides), diazo compounds (such as diazoalkanes, diazoketones, diazoacetates), aliphatic azo compounds, diazirines, ketenes, ketones (including aryl ketones such as acetophenone, benzophenone, anthraquinone, anthrone, quinone, and anthrone-like heterocycles), and peroxy compounds (such as dialkyl peroxides, diacyl peroxides, and peroxyesters). Aryl ketones can be desirable since they are readily capable of undergoing the activation/inactivation/reactivation cycles. As an example, benzophenone is capable of photochemical excitation with the initial formation of an excited singlet state that undergoes intersystem crossing to the triplet state. The excited triplet state can insert into carbon-hydrogen bonds by abstraction of a hydrogen atom (from a substrate, for example), thus creating a radical pair. Subsequent collapse of the radical pair leads to formation of a new carbon-carbon bond. If a reactive bond (e.g., carbon-hydrogen) is not available for bonding, the ultraviolet light-induced excitation of the benzophenone group is reversible and the molecule returns to ground state energy level upon removal of the energy source.

The linking compound can include various types of molecules including polymers and co-polymers. In some embodiments, the linking compound is an amphiphilic polymer. In an embodiment, the linking compound is a copolymer of 1-vinyl-2-pyrrolidone and N-(3-aminopropyl)methacrylamide (APMA) that is derivatized to include one or more latent reactive groups.

In some embodiments, the linking compound can include at least two latent reactive groups. While not intending to be bound by theory, linking molecules with at least two latent reactive groups can offer an advantage in adhering an elution control matrix to a substrate because one latent reactive group can be bound to the surface of the substrate while the other can be bound to the elution control matrix or to other linking molecules.

An elution control matrix 406 can be disposed on the primer layer 404. The elution control matrix 406 may include one or more degradable polymers, one or more non-degradable polymers, or combinations of both. The elution control matrix 406 can also include one or more active agents. The elution control matrix 406 can be configured to control the rate at which the active agent is eluted there from. The thickness of the elution control matrix 406 can depend on many factors including, for example, the specific polymers used in the matrix, the desired loading of active agent within the elution control matrix 406, the type of medical device being coated, etc. In some embodiments, the elution control matrix 406 is from about 0.5 microns to about 200 microns thick.

### Substrates

It will be appreciated that embodiments of the invention can be used in conjunction with various types of substrates. By way of example, embodiments of the invention can be used to increase adhesion of an elution control matrix to a polymeric substrate. Polymeric substrates can include polysiloxanes, polyurethanes, polyamides, polyethylenes, and the like.

In some embodiments, the substrate comprises a polysiloxane. The term “polysiloxane”, as used herein, shall refer to the class of polymers having a silicon-oxygen backbone. Polysiloxanes can be linear, branched, cyclic, or cross-linked. Polysiloxanes can have various side groups attached to the silicon atoms in the backbone including organic groups such as aryl and/or alkyl groups. Exemplary polysiloxanes can include poly(alkylsiloxanes); poly(arylsiloxanes), poly(arylalkylsiloxanes), poly(alkoxysiloxanes), and copolymers thereof such as diphenylsiloxane-dimethylsiloxane copolymers, and combinations thereof. Specific polysiloxanes of the invention include poly(dimethylsiloxane), poly(diethylsiloxane), poly(methylphenylsiloxane), poly(vinylmethylsiloxane), poly(vinylphenylsiloxane), and poly(diphenylsiloxane). Polysiloxanes of the invention can have durometer ratings as is desirable for the specific application. By way of example, polysiloxanes used in some embodiments of the invention can have a durometer rating of about 10 to about 80 Shore A. Polysiloxanes include the class of polymers referred to as “silicones”. Exemplary polysiloxanes can specifically include C6-135, C6-150, C6-165, C6-180, C6-235, C6-250, C6-265, C6-350LH, and various silicones sold under the trade name SILASTIC commercially available from Dow Corning Corporation, Midland, Mich. Polysiloxanes of embodiments herein can specifically include those polysiloxanes referred to as “silicone rubber” by those of skill in the art.

In some embodiments, the substrate includes a material that is clad with a layer of a polysiloxane. By way of example, the substrate could include stainless steel that is clad with a layer of a polysiloxane, such as a silicone. Silicone cladding can offer the advantages of biocompatibility and chemical resistance to an underlying material that may not otherwise enjoy the same properties.

In some embodiments, the surface of the substrate can be treated in various ways to enhance the degree of adhesion thereto. By way of example, the surface of the substrate could be plasma treated, or otherwise chemically modified such as by covalently bonding additional materials to the substrate. However, in other embodiments, the surface of the substrate is untreated before a linking compound or a primer layer of a material such as parylene is applied thereto.

### Medical Devices

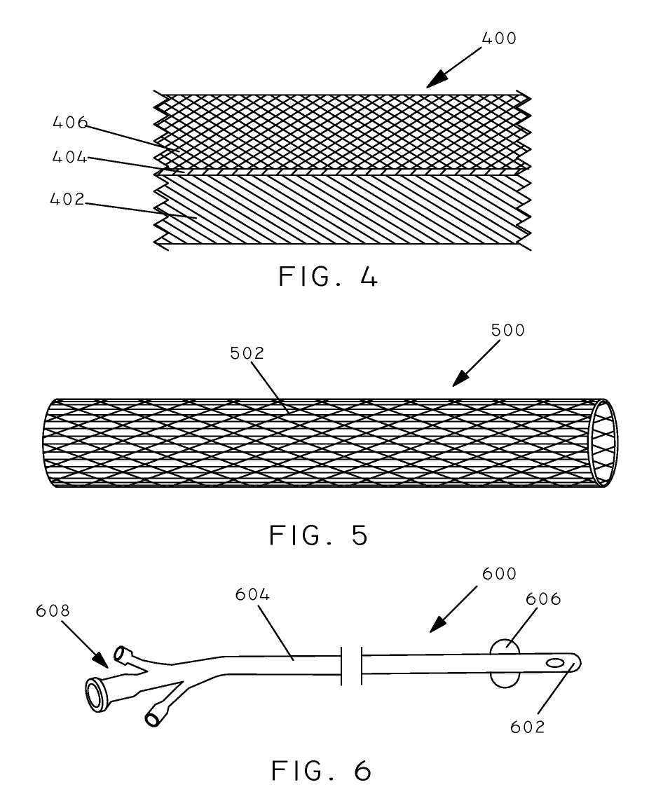

It will be appreciated that embodiments of the invention can be used in conjunction with, and can include, many different types of medical devices. For example, in FIG. 5 a perspective view of a stent 500 is shown in accordance with an embodiment of the invention. The stent 500 is fabricated with a mesh-type construction and includes a plurality of wires or struts 502 that may have a polymeric substrate, such as a polysiloxane, or may have a substrate that includes a metal coated with a polymer. In some embodiments, a parylene layer or a linking compound as described herein can be used to increase adhesion of an elution control matrix to the underlying polymeric substrate.

As a further example of the various medical devices that can be made in accordance with some embodiments of the invention, FIG. 6 shows a schematic view of a Foley-type catheter 600. The catheter 600 can include a tip 602, shaft 604, balloon 606, and proximal end 608. In an embodiment, various portions of the catheter 600 are made of a polysiloxane. A parylene layer or a linking compound, as described herein, can be disposed on one or more of the tip 602, shaft 604, balloon 606, and proximal end 608, and can be used to increase adhesion of an elution control matrix to the underlying polysiloxane substrate. In some embodiments, only certain parts of the catheter 600 are covered with a primer layer and an elution control matrix. In other embodiments, the whole catheter 600 is covered with a primer layer and an elution control matrix.

Embodiments of the invention can be used with both implantable devices and non-implantable medical devices. Embodiments of the invention can be used with implantable, or transitorily implantable, devices including, but not limited to, vascular devices such as grafts (e.g., abdominal aortic aneurysm grafts, etc.), stents (e.g., self-expanding stents typically made from nitinol, balloon-expanded stents typically prepared from stainless steel, degradable coronary stents, etc.), catheters (including arterial, intravenous, blood pressure, stent graft, etc.), valves (e.g., polymeric or carbon mechanical valves, tissue valves, valve designs including percutaneous, sewing cuff, and the like), embolic protection filters (including distal protection devices), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies (e.g., batteries, etc.), peripheral cardiovascular devices, atrial septal defect closures, left atrial appendage filters, valve annuloplasty devices (e.g., annuloplasty rings), mitral valve repair devices, vascular intervention devices, ventricular assist pumps, and vascular access devices (including parenteral feeding catheters, vascular access ports, central venous access catheters); surgical devices such as sutures of all types, staples, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps; orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, implanted drug infusion tubes, drug infusion catheters, and intravitreal drug delivery devices; ophthalmic devices including orbital implants, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices; synthetic prostheses such as breast prostheses and artificial organs (e.g., pancreas, liver, lungs, heart, etc.); respiratory devices including lung catheters; neurological devices such as neurostimulators, neurological catheters, neurovascular balloon catheters, neuro-aneurysm treatment coils, and neuropatches; ear nose and throat devices such as nasal buttons, nasal and airway splints, nasal tampons, ear wicks, ear drainage tubes, tympanostomy vent tubes, otological strips, laryngectomy tubes, esophageal tubes, esophageal stents, laryngeal stents, salivary bypass tubes, and tracheostomy tubes; biosensor devices including glucose sensors, cardiac sensors, intra-arterial blood gas sensors; oncological implants; and pain management implants.

Classes of suitable non-implantable devices can include dialysis devices and associated tubing, catheters, membranes, and grafts; autotransfusion devices; vascular and surgical devices including atherectomy catheters, angiographic catheters, intraaortic balloon pumps, intracardiac suction devices, blood pumps, blood oxygenator devices (including tubing and membranes), blood filters, blood temperature monitors, hemoperfusion units, plasmapheresis units, transition sheaths, dialators, intrauterine pressure devices, clot extraction catheters, percutaneous transluminal angioplasty catheters, electrophysiology catheters, breathing circuit connectors, stylets (vascular and non-vascular), coronary guide wires, peripheral guide wires; dialators (e.g., urinary, etc.); surgical instruments (e.g. scalpels and the like); endoscopic devices (such as endoscopic surgical tissue extractors, esophageal stethoscopes); and general medical and medically related devices including blood storage bags, umbilical tape, membranes, gloves, surgical drapes, wound dressings, wound management devices, needles, percutaneous closure devices, transducer protectors, pessary, uterine bleeding patches, PAP brushes, clamps (including bulldog clamps), cannulae, cell culture devices, materials for in vitro diagnostics, chromatographic support materials, infection control devices, colostomy bag attachment devices, birth control devices; disposable temperature probes; and pledgets.

In some aspects, embodiments of the invention can be utilized in connection with ophthalmic devices. Suitable ophthalmic devices in accordance with these aspects can provide bioactive agent to any desired area of the eye. In some aspects, the devices can be utilized to deliver bioactive agent to an anterior segment of the eye (in front of the lens), and/or a posterior segment of the eye (behind the lens). Suitable ophthalmic devices can also be utilized to provide bioactive agent to tissues in proximity to the eye, when desired.

In some aspects, embodiments of the invention can be utilized in connection with ophthalmic devices configured for placement at an external or internal site of the eye. Suitable external devices can be configured for topical administration of bioactive agent. Such external devices can reside on an external surface of the eye, such as the cornea (for example, contact lenses) or bulbar conjunctiva. In some embodiments, suitable external devices can reside in proximity to an external surface of the eye.

Devices configured for placement at an internal site of the eye can reside within any desired area of the eye. In some aspects, the ophthalmic devices can be configured for placement at an intraocular site, such as the vitreous. Illustrative intraocular devices include, but are not limited to, those described in U.S. Pat. Nos. 6,719,750 B2 (“Devices for Intraocular Drug Delivery,” Varner et al.) and 5,466,233 (“Tack for Intraocular Drug Delivery and Method for Inserting and Removing Same,” Weiner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and U.S. application Ser. Nos. 11/204,195 (filed Aug. 15, 2005, Anderson et al.), 11/204,271 (filed Aug. 15, 2005, Anderson et al.), 11/203,981 (filed Aug. 15, 2005, Anderson et al.), 11/203,879 (filed Aug. 15, 2005, Anderson et al.), 11/203,931 (filed Aug. 15, 2005, Anderson et al.); and related applications.

In some aspects, the ophthalmic devices can be configured for placement at a subretinal area within the eye. Illustrative ophthalmic devices for subretinal application include, but are not limited to, those described in U.S. Patent Publication No. 2005/0143363 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); U.S. application Ser. No. 11/175,850 (“Methods and Devices for the Treatment of Ocular Conditions,” de Juan et al.); and related applications.

Suitable ophthalmic devices can be configured for placement within any desired tissues of the eye. For example, ophthalmic devices can be configured for placement at a subconjunctival area of the eye, such as devices positioned extrasclerally but under the conjunctiva, such as glaucoma drainage devices and the like.

### Degradable Polymers

In an embodiment, the elution control matrix can include one or more degradable polymers. The term “degradable” as used herein with reference to polymers, shall refer to those natural or synthetic polymers that break down under physiological conditions into constituent components over a period of time. By way of example, many degradable polymers include hydrolytically unstable linkages in the polymeric backbone. The cleavage of these unstable linkages leads to degradation of the polymer. The terms “erodible”, “bioerodible”, “biodegradable” and “non-durable” shall be used herein interchangeably with the term “degradable”. Degradable polymers can include both natural and synthetic polymers. Degradable polymers of the invention can include both those with bulk erosion characteristics and those with surface erosion characteristics.

Synthetic degradable polymers can include: degradable polyesters (such as poly(glycolic acid), poly(lactic acid), poly(lactic-co-glycolic acid), poly(dioxanone), polylactones (e.g., poly(caprolactone)), poly(3-hydroxybutyrate), poly(3-hydroxyvalerate), poly(valerolactone), poly(tartronic acid), poly(B-malonic acid), poly(propylene fumarate)); degradable polyesteramides; degradable polyanhydrides (such as poly(sebacic acid), poly(1,6-bis(carboxyphenoxy)hexane, poly(1,3-bis(carboxyphenoxy)propane); degradable polycarbonates (such as tyrosine-based polycarbonates); degradable polyiminocarbonates; degradable polyarylates (such as tyrosine-based polyarylates); degradable polyorthoesters; degradable polyurethanes; degradable polyphosphazenes; and degradable polyhydroxyalkanoates; and copolymers thereof.

Natural or naturally-based degradable polymers can include polysaccharides and modified polysaccharides such as starch, cellulose, chitin, chitosan, and copolymers thereof.

Specific examples of degradable polymers include poly(ether ester) multiblock copolymers based on poly(ethylene glycol) (PEG) and poly(butylene terephthalate) that can be described by the following general structure:

[—(OCH₂CH₂)ₙ—O—C(O)—C₆H₄—C(O)-]x[-O—(CH₂)₄—O—C(O)—C₆H₄—C(O)-]y,

where —C₆H₄— designates the divalent aromatic ring residue from each esterified molecule of terephthalic acid, n represents the number of ethylene oxide units in each hydrophilic PEG block, x represents the number of hydrophilic blocks in the copolymer, and y represents the number of hydrophobic blocks in the copolymer. n can be selected such that the molecular weight of the PEG block is between about 300 and about 4000. X and y can be selected so that the multiblock copolymer contains from about 55% up to about 80% PEG by weight. The block copolymer can be engineered to provide a wide array of physical characteristics (e.g., hydrophilicity, adherence, strength, malleability, degradability, durability, flexibility) and active agent release characteristics (e.g., through controlled polymer degradation and swelling) by varying the values of n, x and y in the copolymer structure.

Degradable polyesteramides can include those formed from the monomers OH-x-OH, z, and COOH-y-COOH, wherein x is alkyl, y is alkyl, and z is leucine or phenylalanine.

Degradable polymeric materials can also be selected from: (a) non-peptide polyamino polymers; (b) polyiminocarbonates; (c) amino acid-derived polycarbonates and polyarylates; and (d) poly(alkylene oxide) polymers.

In an embodiment, the degradable polymeric material is composed of a non-peptide polyamino acid polymer. Exemplary non-peptide polyamino acid polymers are described, for example, in U.S. Pat. No. 4,638,045 (“Non-Peptide Polyamino Acid Bioerodible Polymers,” Jan. 20, 1987). Generally speaking, these polymeric materials are derived from monomers, including two or three amino acid units having one of the following two structures illustrated below:

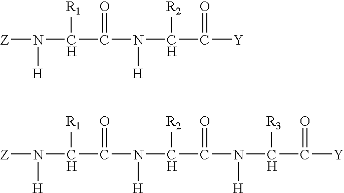

wherein the monomer units are joined via hydrolytically labile bonds at not less than one of the side groups R₁, R₂, and R₃, and where R₁, R₂, R₃ are the side chains of naturally occurring amino acids; Z is any desirable amine protecting group or hydrogen; and Y is any desirable carboxyl protecting group or hydroxyl. Each monomer unit comprises naturally occurring amino acids that are then polymerized as monomer units via linkages other than by the amide or “peptide” bond. The monomer units can be composed of two or three amino acids united through a peptide bond and thus comprise dipeptides or tripeptides. Regardless of the precise composition of the monomer unit, all are polymerized by hydrolytically labile bonds via their respective side chains rather than via the amino and carboxyl groups forming the amide bond typical of polypeptide chains. Such polymer compositions are nontoxic, are degradable, and can provide zero-order release kinetics for the delivery of active agents in a variety of therapeutic applications. According to these aspects, the amino acids are selected from naturally occurring L-alpha amino acids, including alanine, valine, leucine, isoleucine, proline, serine, threonine, aspartic acid, glutamic acid, asparagine, glutamine, lysine, hydroxylysine, arginine, hydroxyproline, methionine, cysteine, cystine, phenylalanine, tyrosine, tryptophan, histidine, citrulline, ornithine, lanthionine, hypoglycin A, β-alanine, γ-amino butyric acid, a aminoadipic acid, canavanine, venkolic acid, thiolhistidine, ergothionine, dihydroxyphenylalanine, and other amino acids well recognized and characterized in protein chemistry.

Degradable polymers of the invention can also include polymerized polysaccharides such as those described in U.S. Publ. Pat. Application No. 2005/0255142, entitled “COATINGS FOR MEDICAL ARTICLES INCLUDING NATURAL BIODEGRADABLE POLYSACCHARIDES”, U.S. Publ. Pat. Application No. 2007/0065481, entitled “COATINGS INCLUDING NATURAL BIODEGRADABLE POLYSACCHARIDES AND USES THEREOF”, and in U.S. Application No. 60/782,957, entitled “HYDROPHOBIC DERIVATIVES OF NATURAL BIODEGRADABLE POLYSACCHARIDES”, all of which are herein incorporated by reference.

Degradable polymers of the invention can also include dextran based polymers such as those described in U.S. Pat. No. 6,303,148, entitled “PROCESS FOR THE PREPARATION OF A CONTROLLED RELEASE SYTEM”. Exemplary dextran based degradable polymers including those available commercially under the trade name OCTODEX.

Degradable polymers of the invention can further include collagen/hyaluronic acid polymers.

Degradable polymers of the invention can include multi-block copolymers, comprising at least two hydrolysable segments derived from pre-polymers A and B, which segments are linked by a multi-functional chain-extender and are chosen from the pre-polymers A and B, and triblock copolymers ABA and BAB, wherein the multi-block copolymer is amorphous and has one or more glass transition temperatures (Tg) of at most 37° C. (Tg) at physiological (body) conditions. The pre-polymers A and B can be a hydrolysable polyester, polyetherester, polycarbonate, polyestercarbonate, polyanhydride or copolymers thereof, derived from cyclic monomers such as lactide (L,D or L/D), glycolide, ε-caprolactone, δ-valerolactone, trimethylene carbonate, tetramethylene carbonate, 1,5-dioxepane-2-one, 1,4-dioxane-2-one (para-dioxanone) or cyclic anhydrides (oxepane-2,7-dione). The composition of the pre-polymers can be chosen in such a way that the maximum glass transition temperature of the resulting copolymer is below 37° C. at body conditions. To fulfill the requirement of a Tg below 37° C., some of the above-mentioned monomers or combinations of monomers can be more preferred than others. This may by itself lower the Tg, or the pre-polymer is initiated with a polyethylene glycol with sufficient molecular weight to lower the glass transition temperature of the copolymer. The degradable multi-block copolymers can include hydrolysable sequences being amorphous and the segments can be linked by a multifunctional chain-extender, the segments having different physical and degradation characteristics. For example, a multi-block co-polyester consisting of a glycolide-ε-caprolactone segment and a lactide-glycolide segment can be composed of two different polyester pre-polymers. By controlling the segment monomer composition, segment ratio and length, a variety of polymers with properties that can easily be tuned can be obtained.

### Non-Degradable Polymers

Embodiments of the invention can include one or more non-degradable (durable) polymers in the elution control matrix. In an embodiment, the non-degradable polymer includes a plurality of polymers, including a first polymer and a second polymer. When the elution control matrix includes only one non-degradable polymer, it can be either a first or second polymer as described herein. As used herein, term “(meth)acrylate” when used in describing polymers shall mean the form including the methyl group (methacrylate) or the form without the methyl group (acrylate).

First polymers of the invention can include a polymer selected from the group consisting of poly(alkyl(meth)acrylates) and poly(aromatic(meth)acrylates), where “(meth)” will be understood by those skilled in the art to include such molecules in either the acrylic and/or methacrylic form (corresponding to the acrylates and/or methacrylates, respectively). An exemplary first polymer is poly(n-butyl methacrylate) (pBMA). Such polymers are available commercially, e.g., from Aldrich, with molecular weights ranging from about 200,000 Daltons to about 320,000 Daltons, and with varying inherent viscosity, solubility, and form (e.g., as crystals or powder). In some embodiments, poly(n-butyl methacrylate) (PBMA) is used with a molecular weight of about 200,000 Daltons to about 300,000 Daltons.

Examples of suitable first polymers also include polymers selected from the group consisting of poly(aryl(meth)acrylates), poly(aralkyl (meth)acrylates), and poly(aryloxyalkyl(meth)acrylates). Such terms are used to describe polymeric structures wherein at least one carbon chain and at least one aromatic ring are combined with acrylic groups, typically esters, to provide a composition. In particular, exemplary polymeric structures include those with aryl groups having from 6 to 16 carbon atoms and with weight average molecular weights from about 50 to about 900 kilodaltons. Suitable poly(aralkyl(meth)acrylates), poly(arylalky(meth)acrylates) or poly(aryloxyalkyl (meth)acrylates) can be made from aromatic esters derived from alcohols also containing aromatic moieties. Examples of poly(aryl(meth)acrylates) include poly(9-anthracenyl methacrylate), poly(chlorophenylacrylate), poly(methacryloxy-2-hydroxybenzophenone), poly(methacryloxybenzotriazole), poly(naphthylacrylate) and -methacrylate), poly(4-nitrophenyl acrylate), poly(pentachloro(bromo, fluoro) acrylate) and -methacrylate), and poly(phenyl acrylate) and -methacrylate). Examples of poly(aralkyl (meth)acrylates) include poly(benzyl acrylate) and -methacrylate), poly(2-phenethyl acrylate) and -methacrylate, and poly(1-pyrenylmethyl methacrylate). Examples of poly(aryloxyalkyl (meth)acrylates) include poly(phenoxyethyl acrylate) and -methacrylate), and poly(polyethylene glycol phenyl ether acrylates) and -methacrylates with varying polyethylene glycol molecular weights.

Examples of suitable second polymers are available commercially and include poly(ethylene-co-vinyl acetate) (pEVA) having vinyl acetate concentrations of between about 10% and about 50% (12%, 14%, 18%, 25%, 33% versions are commercially available), in the form of beads, pellets, granules, etc. The pEVA co-polymers with lower percent vinyl acetate become increasingly insoluble in typical solvents, whereas those with higher percent vinyl acetate become decreasingly durable.

An exemplary polymer mixture includes mixtures of pBMA and pEVA. This mixture of polymers can be used with absolute polymer concentrations (i.e., the total combined concentrations of both polymers in the coating material), of between about 0.25 wt. % and about 99 wt. %. This mixture can also be used with individual polymer concentrations in the coating solution of between about 0.05 wt. % and about 99 wt. %. In one embodiment the polymer mixture includes pBMA with a molecular weight of from 100 kilodaltons to 900 kilodaltons and a pEVA copolymer with a vinyl acetate content of from 24 to 36 weight percent. In an embodiment the polymer mixture includes pBMA with a molecular weight of from 200 kilodaltons to 300 kilodaltons and a pEVA copolymer with a vinyl acetate content of from 24 to 36 weight percent. The concentration of the active agent or agents dissolved or suspended in the coating mixture can range from 0.01 to 99 percent, by weight, based on the weight of the final coating material.

Second polymers can also comprise one or more polymers selected from the group consisting of (i) poly(alkylene-co-alkyl(meth)acrylates, (ii) ethylene copolymers with other alkylenes, (iii) polybutenes, (iv) diolefin derived non-aromatic polymers and copolymers, (v) aromatic group-containing copolymers, and (vi) epichlorohydrin-containing polymers.

Poly(alkylene-co-alkyl(meth)acrylates) include those copolymers in which the alkyl groups are either linear or branched, and substituted or unsubstituted with non-interfering groups or atoms. Such alkyl groups can comprise from 1 to 8 carbon atoms, inclusive. Such alkyl groups can comprise from 1 to 4 carbon atoms, inclusive. In an embodiment, the alkyl group is methyl. In some embodiments, copolymers that include such alkyl groups can comprise from about 15% to about 80% (wt) of alkyl acrylate. When the alkyl group is methyl, the polymer contains from about 20% to about 40% methyl acrylate in some embodiments, and from about 25% to about 30% methyl acrylate in a particular embodiment. When the alkyl group is ethyl, the polymer contains from about 15% to about 40% ethyl acrylate in an embodiment, and when the alkyl group is butyl, the polymer contains from about 20% to about 40% butyl acrylate in an embodiment.

Alternatively, second polymers can comprise ethylene copolymers with other alkylenes, which in turn, can include straight and branched alkylenes, as well as substituted or unsubstituted alkylenes. Examples include copolymers prepared from alkylenes that comprise from 3 to 8 branched or linear carbon atoms, inclusive. In an embodiment, copolymers prepared from alkylene groups that comprise from 3 to 4 branched or linear carbon atoms, inclusive. In a particular embodiment, copolymers prepared from alkylene groups containing 3 carbon atoms (e.g., propene). By way of example, the other alkylene is a straight chain alkylene (e.g., 1-alkylene). Exemplary copolymers of this type can comprise from about 20% to about 90% (based on moles) of ethylene. In an embodiment, copolymers of this type comprise from about 35% to about 80% (mole) of ethylene. Such copolymers will have a molecular weight of between about 30 kilodaltons to about 500 kilodaltons. Exemplary copolymers are selected from the group consisting of poly(ethylene-co-propylene), poly(ethylene-co-1-butene), polyethylene-co-1-butene-co-1-hexene) and/or poly(ethylene-co-1-octene).

“Polybutenes” include polymers derived by homopolymerizing or randomly interpolymerizing isobutylene, 1-butene and/or 2-butene. The polybutene can be a homopolymer of any of the isomers or it can be a copolymer or a terpolymer of any of the monomers in any ratio. In an embodiment, the polybutene contains at least about 90% (wt) of isobutylene or 1-butene. In a particular embodiment, the polybutene contains at least about 90% (wt) of isobutylene. The polybutene may contain non-interfering amounts of other ingredients or additives, for instance it can contain up to 1000 ppm of an antioxidant (e.g., 2,6-di-tert-butyl-methylphenol). By way of example, the polybutene can have a molecular weight between about 150 kilodaltons and about 1,000 kilodaltons. In an embodiment, the polybutene can have between about 200 kilodaltons and about 600 kilodaltons. In a particular embodiment, the polybutene can have between about 350 kilodaltons and about 500 kilodaltons. Polybutenes having a molecular weight greater than about 600 kilodaltons, including greater than 1,000 kilodaltons are available but are expected to be more difficult to work with.

Additional alternative second polymers include diolefin-derived, non-aromatic polymers and copolymers, including those in which the diolefin monomer used to prepare the polymer or copolymer is selected from butadiene (CH₂═CH—CH═CH₂) and/or isoprene (CH₂═CH—C(CH₃)═CH₂). In an embodiment, the polymer is a homopolymer derived from diolefin monomers or is a copolymer of diolefin monomer with non-aromatic mono-olefin monomer, and optionally, the homopolymer or copolymer can be partially hydrogenated. Such polymers can be selected from the group consisting of polybutadienes prepared by the polymerization of cis-, trans- and/or 1,2-monomer units, or from a mixture of all three monomers, and polyisoprenes prepared by the polymerization of cis-1,4- and/or trans-1,4-monomer units. Alternatively, the polymer is a copolymer, including graft copolymers, and random copolymers based on a non-aromatic mono-olefin monomer such as acrylonitrile, and an alkyl (meth)acrylate and/or isobutylene. In an embodiment, when the mono-olefin monomer is acrylonitrile, the interpolymerized acrylonitrile is present at up to about 50% by weight; and when the mono-olefin monomer is isobutylene, the diolefin is isoprene (e.g., to form what is commercially known as a “butyl rubber”). Exemplary polymers and copolymers have a molecular weight between about 150 kilodaltons and about 1,000 kilodaltons. In an embodiment, polymers and copolymers have a molecular weight between about 200 kilodaltons and about 600 kilodaltons.

Additional alternative second polymers include aromatic group-containing copolymers, including random copolymers, block copolymers and graft copolymers. In an embodiment, the aromatic group is incorporated into the copolymer via the polymerization of styrene. In a particular embodiment, the random copolymer is a copolymer derived from copolymerization of styrene monomer and one or more monomers selected from butadiene, isoprene, acrylonitrile, a C₁-C₄ alkyl (meth)acrylate (e.g., methyl methacrylate) and/or butene. Useful block copolymers include copolymer containing (a) blocks of polystyrene, (b) blocks of an polyolefin selected from polybutadiene, polyisoprene and/or polybutene (e.g., isobutylene), and (c) optionally a third monomer (e.g., ethylene) copolymerized in the polyolefin block. The aromatic group-containing copolymers contain about 10% to about 50% (wt.) of polymerized aromatic monomer and the molecular weight of the copolymer is from about 300 kilodaltons to about 500 kilodaltons. In an embodiment, the molecular weight of the copolymer is from about 100 kilodaltons to about 300 kilodaltons.

Additional alternative second polymers include epichlorohydrin homopolymers and poly(epichlorohydrin-co-alkylene oxide) copolymers. In an embodiment, in the case of the copolymer, the copolymerized alkylene oxide is ethylene oxide. By way of example, epichlorohydrin content of the epichlorohydrin-containing polymer is from about 30% to 100% (wt). In an embodiment, epichlorohydrin content is from about 50% to 100% (wt). In an embodiment, the epichlorohydrin-containing polymers have a molecular weight from about 100 kilodaltons to about 300 kilodaltons.

Non-degradable polymers can also include those described in U.S. Pat. App. No. 60/703,555, entitled “DEVICES, ARTICLES, COATINGS, AND METHODS FOR CONTROLLED ACTIVE AGENT RELEASE OR HEMOCOMPATIBILITY”, the contents of which is herein incorporated by reference. As a specific example, non-degradable polymers can include random copolymers of butyl methacrylate-co-acrylamido-methyl-propane sulfonate (BMA-AMPS). In some embodiments, the random copolymer can include AMPS in an amount equal to about 0.5 mol. % to about 40 mol. %.

### Hydrophobic Polymers

Embodiments of the invention can include one or more hydrophobic polymers in the elution control matrix. Hydrophobic polymers can be either degradable or non-degradable. One method of defining the hydrophobicity of a polymer is by the solubility parameter (or Hildebrand parameter) of the polymer. The solubility parameter describes the attractive strength between molecules of the material. The solubility parameter is represented by Equation 1:

δ=(ΔE^{v}/V)^{1/2} (Equation 1)

where δ=solubility parameter ((cal/cm³)^{1/2})

ΔE^{v}=energy of vaporization (cal)

V=molar volume (cm³)

Solubility parameters cannot be calculated for polymers from heat of vaporization data because of their nonvolatility. Accordingly, solubility parameters must be calculated indirectly. One method involves identifying solvents in which a polymer dissolves without a change in heat or volume and then defining the solubility parameter of the polymer to be the same as the solubility parameters of the identified solvents. A more complete discussion of solubility parameters and methods of calculating the same can be found in Brandup et al., Polymer Handbook, 4th Ed., John Wiley & Sons, N.Y. (1999) beginning at VII p. 675.

As a general rule, the value of the solubility parameter δ is inversely proportional to the degree of hydrophobicity of a polymer. Thus, polymers that are very hydrophobic may have a low solubility parameter value. This general proposition is particularly applicable for polymers having a glass transition temperature below physiological temperature. In an embodiment, polymers used with the invention have a solubility parameter less than about 11.0 (cal/cm³)^{1/2}. In an embodiment polymers used with the invention have a solubility parameter of less than about 10.0 (cal/cm³)^{1/2}.

### Silane Compounds

In an embodiment, the primer layer can include a silane compound, a hydrolysis (or solvolysis) reaction product of the silane compound, a polymeric reaction product formed from the hydrolysis reaction product of the silane compound, or a combination thereof. Chlorine, nitrogen, alkyloxy groups, or acetoxy groups coupling directly to silicon can produce chlorosilanes, silylamines (silazanes), alkoxysilanes, and acyloxysilanes respectively. Silane compounds of the invention can include these types of reactive silane moieties. In an embodiment, the silane compound can have one or more tri(C₁-C₃)alkoxysilyl groups. Suitable groups include trimethoxysilyl, triethoxysilyl, and tripropoxysilyl, and combinations thereof. In some embodiments, the silane compound has at least two trimethoxysilyl groups. In an embodiment, the silane compound is 1,4-bis(trimethoxysilyethyl)benzene.

The present invention may be better understood with reference to the following examples. These examples are intended to be representative of specific embodiments of the invention, and are not intended as limiting the scope of the invention.

### EXAMPLES

### Example 1

### Adhering an Elution Control Matrix to a Polysiloxane with a Parylene Layer

An elution control coating solution was formed by combining poly-n-butylmethacrylate (PBMA) and polyethylene-co-vinyl acetate (PEVA) in a solvent of THF to reach a concentration of 15 mg/mL PBMA and 15 mg/mL PEVA (total solids concentration of 30 mg/mL).

Treatment A: PBMA/PEVA Matrix on Polysiloxane Substrate

For Treatment A, a piece of polysiloxane was first thoroughly cleaned by wiping it down with a solution of isopropyl alcohol (IPA). After the residual IPA was fully evaporated, the polysiloxane material was then dipped into the elution control coating solution formed as described above. The coated polysiloxane material was then allowed to dry for approximately thirty minutes under ambient conditions.

Treatment B: PBMA/PEVA Matrix on Roughened Polysiloxane Substrate

For Treatment B, a piece of a polysiloxane was first thoroughly cleaned by wiping it down with a solution of isopropyl alcohol (IPA). The residual IPA was then allowed to evaporate off. Next, a fine emery cloth was used to roughen the surface of the polysiloxane by rubbing it for approximately 30 seconds to 60 seconds. The polysiloxane material was then dipped into the elution control coating solution formed as described above. The coated polysiloxane material was then allowed to dry for approximately thirty minutes under ambient conditions.

Treatment C: PBMA/PEVA Matrix on Parylene Pretreated Polysiloxane Substrate

For Treatment C, a piece of polysiloxane was first thoroughly cleaned by wiping it down with a solution of isopropyl alcohol (IPA). The residual IPA was then allowed to evaporate off. Next, a layer of parylene C was then vapor deposited onto the polysiloxane. Specifically, 0.5 grams of parylene C dimer (Specialty Coating Systems, Indianapolis, Ind.) was loaded into a vapor deposition system PDS-2010 LABCOTER® (Specialty Coating Systems, Indianapolis, Ind.). A coating cycle was then initiated and a layer of parylene approximately 0.2-0.3 microns thick was deposited onto the polysiloxane substrate under vacuum. The coated polysiloxane substrate was then removed from the parylene coating apparatus.

Next, the parylene coated polysiloxane substrate was dipped into the elution control coating solution formed as described above. The coated polysiloxane material was then allowed to dry for approximately thirty minutes under ambient conditions.

Treatment D: PBMA/PEVA Matrix on Stainless Steel Substrate

For Treatment D, a stainless steel coupon was first thoroughly cleaned by wiping it down with a solution of isopropyl alcohol (IPA). After the residual IPA was fully evaporated, the stainless steel coupon was then dipped into the elution control coating solution formed as described above. The coated stainless steel coupon was then allowed to dry for approximately thirty minutes under ambient conditions.

### Example 2

### Evaluation of Adhesion of Treatments A-D

The degree of adhesion between the elution control matrix and the substrate was the evaluated for each of the test treatments described in Example 1. Adhesion was assessed using two different tests, referred to herein as a “tweezer” test and a “peel” test.

A. Tweezer Test Procedure

A metal tweezer instrument have a semi-sharp tip was applied against the surface of the coating to be tested and then dragged while maintaining constant pressure for a distance of about 1 centimeter. The furrow created by dragging the tweezer tip was then inspected using optical microscopy to determine whether or not the coating had formed loose flaps surrounding the furrow. The tweezer instrument was then used to pull on any flaps present to determine if the coating could be further separated from the substrate. The detection of flaps under optical microscopy was judged as a failing tweezer test. The absence of flaps under optical microscopy was judged as a passing tweezer test.

B. Peel Test Procedure

A metal razor blade was used to score the surface of a coating in a cross-hatch pattern with an average distance between blade passes of about 2 mm. Adhesive labeling tape (Time Med Labeling Systems, Inc., Burr Ridge, Ill.) was then affixed to the scored coating surface and firmly seated by uniformly applying hand pressure. The adhesive labeling tape was then pulled off from the coating surface by pulling at a 90 degree angle to the surface. The coating was then inspected using optical microscopy to assess whether or not any of the coating had dislodged from the substrate. The dislodgement of the coating material from the substrate was judged as a failing peel test. If no coating material was dislodged from the substrate by this procedure, the test was judged as passing.

Treatments A-D, as described in Example 1 above, were subjected to both the tweezer and peel tests. The results are summarized in Table 1 below.

TABLE 1

| Treatment | Tweezer Test | Peel Test |
|---|---|---|
| A | Fail | Fail |
| B | Fail | Fail |
| C | Pass | Pass |
| D | Pass | Pass |

The only distinction between Treatment A and Treatment D was the substrate material (polysiloxane vs. stainless steel respectively). Treatment A failed both the tweezer test and the peel test while Treatment D passed both the tweezer test and the peel test. Accordingly, comparison of the performance of Treatment A (polysiloxane substrate) with the performance of Treatment D (stainless steel substrate) shows that issues associated with insufficient adhesion are particularly acute in the context of polymer substrates, such as polysiloxane, in contrast to other common medical device substrates, such as stainless steel.

Treatment B (roughened polysiloxane surface) failed both the tweezer test and the peel test. This shows that the problem of insufficient adhesion of elution control matrices to polymeric substrates such as a polysiloxane cannot be solved simply by roughening the surface of the substrate.

Treatment C (parylene primer coat) passed both the tweezer test and the peel test. Thus, this example shows that a parylene primer coat can be used to increase adhesion between elution control matrices and polymer substrates.

### Example 3

### Adhering an Elution Control Matrix to a Polysiloxane with a Primer Layer Including Latent Reactive Groups

An elution control coating solution was formed by combining poly-n-butylmethacrylate (PBMA) and polyethylene-co-vinyl acetate (PEVA) in a solvent of THF to reach a concentration of 15 mg/mL PBMA and 15 mg/mL PEVA (total solids concentration of 30 mg/mL).

A linking compound (photo-PVP-APMA) was formed by copolymerization of 1-vinyl-2-pyrrolidone and N-(3-aminopropyl)methacrylamide (APMA) followed by photo-derivatization of the polymer using 4-benzoylbenzoyl chloride under Schotten-Baumann conditions. The unreacted amines of the photopolymer were further acetylated using acetic anhydride to give acetylated photo-PVP-APMA. A linking solution was formed by dissolving acetylated photo-PVP-APMA in water at a concentration of 5 mg/ml.

Treatment E: PBMA/PEVA Matrix on Pretreated Polysiloxane Substrate (Single Illumination)

A piece of polysiloxane was wiped with isopropyl alcohol and dried. The polysiloxane was then completely submerged in the linking solution for approximately one to five minutes. The polysiloxane was then illuminated with UV light for approximately 60 seconds while still submerged in the linking solution (Dymax Bluewave 200 at 3-5 mW/cm²). The polysiloxane was then removed from the linking solution and rinsed thoroughly with water.

Next, the polysiloxane substrate was dipped into the elution control coating solution formed as described above. The coated polysiloxane material was then allowed to dry for approximately thirty minutes under ambient conditions.

Treatment F: PBMA/PEVA Matrix on Pretreated Polysiloxane Substrate (Double Illumination)

A piece of polysiloxane was wiped with isopropyl alcohol and dried. The polysiloxane was then completely submerged in the linking solution for approximately one to five minutes. The polysiloxane was then illuminated with UV light for approximately 60 seconds while still submerged in the linking solution (Dymax Bluewave 200 at 3-5 mW/cm²). The polysiloxane was then removed from the linking solution and rinsed thoroughly with water.

Next, the polysiloxane substrate was dipped into the elution control coating solution formed as described above. The coated polysiloxane material was then allowed to dry for approximately thirty minutes under ambient conditions.

The dried coated polysiloxane substrate was then illuminated with UV light again for approximately 60 seconds (Dymax Bluewave 200 at 3-5 mW/cm²).

Treatment G: PBMA/PEVA Matrix on Polysiloxane Substrate with UV Irradiation

For Treatment G, a piece of polysiloxane was first thoroughly cleaned by wiping it down with a solution of isopropyl alcohol (IPA). After the residual IPA was fully evaporated, the polysiloxane material was then dipped into the elution control coating solution formed as described above. After the polysiloxane was withdrawn, it was allowed to dry for approximately thirty minutes under ambient condition. The polysiloxane was then illuminated with UV light for approximately 60 seconds (Dymax Bluewave 200 at 3-5 mW/cm²).

### Example 4

### Evaluation of Adhesion of Treatments E-G

The degree of adhesion between the elution control matrix and the substrate was evaluated for each of the test treatments described in Example 3. Adhesion was assessed using the “tweezer” test and “peel” tests as described in Example 2 above. The results are shown in Table 2 below.

TABLE 2

| Treatment | Tweezer Test | Peel Test |
|---|---|---|
| E | Pass | Fail |
| F | Pass | Pass |
| G | Fail | Fail |

Treatment G did not contain any photo-reactive linking entity (acetylated photo-PVP-APMA) and thus served as a control to show that UV illumination by itself was not responsible for increased adhesion. Comparing Treatment E (linker compound with single illumination) with Treatment F (linker compound with double illumination) shows that an additional illumination step can enhance the adhesion between the elution control matrix and the substrate. However, even a single illumination treatment (Treatment E) was sufficient to pass the tweezer test. Taken together, this example shows that photoactivatable linking molecules (such as acetylated photo-PVP-APMA) can be used to adhere drug elution matrices to polymeric substrates (such as a polysiloxane).

### Example 5

### Adhering a Coating to a Polysiloxane Substrate with a Silane Compound

A silane compound (1,4-Bis(trimethoxysilyethyl)benzene) was purchased from UCT, Bristol Pa. A silane solution was then formed by dissolving the 1,4-Bis(trimethoxysilyethyl)benzene in isopropanol to a concentration of 0.5% by weight.

Photo-PVP-APMA was formed as described above in Example 3. Polyvinylpyrrolidone (PVP) (K30—m.w. ˜40,000 and K90—m.w. ˜900,000) was obtained from BASF, Florham Park, N.J. A dibenzodisulfonate photo cross-linker (4,5-bis(4-benzoylphenylmethyleneoxy) benzene-1,3-disulfonic acid disodium salt) was prepared according to the method described in example number 1 of U.S. Pat. No. 7,138,541. A coating solution was then formed by dissolving the photo PVP-APMA, PVP K-90, PVP K-30 and the dibenzodisulfonate photo cross-linker at concentrations of respectively, 10 g/L, 20 g/L, 40 g/L and 1.5 g/L in a solvent of 25% Isopropanol and 75% water.

Silicone catheters (16 Fr Bardex, C. R. Bard, Murray Hill, N.J.) were cut up into pieces (n=4) 15 centimeters in length. Four pieces were wiped with isopropyl alcohol. Two pieces (test set) were then dipped into the silane solution at a rate of two centimeters per second with a thirty second dwell time and then pulled out at one centimeter per second. The two coated pieces were air dried for five minutes and then heat cured at 60 degrees Celsius for five minutes. The other two pieces (control set) were simply dried under ambient conditions.

The coating solution was then applied to all four pieces. Specifically, all of the pieces were dipped into the coating solution at a rate of two centimeters per second with a thirty second dwell time and then pulled out at a rate of one centimeter per second. After being air dried for five minutes, all four pieces were UV treated. Specifically, the pieces were suspended midway between opposed ELC 4000 lamps (Electro-Lite Corp., Danbury, Conn.), approximately 40 cm apart, and containing 400 watt mercury vapor bulbs which put out 1.0 mW/cm² from 330-340 nm at the distance of illumination.

All four pieces were then rubbed using a wet laboratory glove. The control set was observed to be less slippery than the test set of pieces, indicating that the control set did not retain as much of the polymers from the coating solution. All pieces were then stained with Congo red. The control set showed very spotty staining, indicating relatively poor coating coverage. The test pieces showed intense and uniform staining, indicating uniform coating coverage.

A 500 gram vertical pinch test was then performed using silicone pads and a vertical pinch testing device. The control set exhibited average frictional forces of about 220 grams. The test set exhibited average frictional forces of less than 15 grams. This example shows that a silane compound such as 1,4-bis(trimethoxysilyethyl)benzene can be used to effectively adhere a polymeric coating, which can be used as an elution matrix, to a silicone substrate.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration to. The phrase “configured” can be used interchangeably with other similar phrases such as arranged and configured, constructed and arranged, constructed, manufactured and arranged, and the like.

The invention has been described with reference to various specific and preferred embodiments and techniques. However, it should be understood that many variations and modifications may be made while remaining within the spirit and scope of the invention.

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

1. A medical device comprising:
a substrate having a surface, the substrate comprising a polysiloxane;
a parylene layer contacting the surface of the substrate; and
an elution control matrix contacting the parylene layer, the elution control matrix comprising a polymeric matrix and an active agent dispersed within the polymeric matrix.

2. The medical device of claim 1, the parylene layer having a thickness of between about 0.01 microns to about 1.0 micron.

3. The medical device of claim 1, the elution control matrix adhered to the substrate sufficiently to resist peeling more than an otherwise identical elution control matrix disposed directly on a polysiloxane polymer.

4. The medical device of claim 1, the elution control matrix configured to contact bodily fluids when the device is positioned within a patient.

5. The medical device of claim 1, the parylene comprising poly(2-chloro-paraxylylene).

6. The medical device of claim 1, the elution control matrix having a thickness of about 0.5 microns to about 200 microns.

7. The medical device of claim 1, the polymeric matrix comprising a degradable polymer.

8. The medical device of claim 1, the polymeric matrix comprising a non-degradable polymer.

9. The medical device of claim 1, the polymeric matrix comprising poly(n-butylmethacrylate) and poly(ethylene-co-vinyl)acetate.

10. The medical device of claim 1, the first polymer comprising polybutadiene and the second polymer comprising poly(n-butylmethacrylate).

11. The medical device of claim 1, the polymeric matrix comprising a polymer with a solubility parameter less than about 11.0 (cal/cm³)^{1/2}.

12. A medical device comprising:
a substrate having a surface, the substrate comprising a polymer;
a parylene layer contacting the surface of the substrate, the parylene layer having a thickness of between about 0.01 microns to about 1.0 micron; and
an elution control matrix contacting the parylene layer, the elution control matrix comprising a polymeric matrix and an active agent dispersed within the polymeric matrix.

13. The medical device of claim 12, the substrate comprising a polymer with a water contact angle of greater than about 50 degrees.

14. A method of bonding an elution control matrix to a substrate surface comprising:
depositing a parylene layer on the substrate surface, the substrate comprising a polysiloxane;
depositing an elution control matrix on the parylene layer, the elution control matrix comprising an active agent.

15. The method of claim 14, the parylene layer having a thickness of between about 0.01 microns to about 1.0 micron.

16. The method of claim 14, the elution control matrix comprising poly(n-butylmethacrylate) and poly(ethylene-co-vinyl)acetate.

17. A medical device comprising:
a substrate, the substrate comprising a polysiloxane;
a silane compound bonded to the substrate; and
a polymer layer bonded to the silane compound through the residue of one or more latent reactive groups.

18. The medical device of claim 17, the silane compound comprising 1,4-bis(trimethoxysilyethyl)benzene.

19. The medical device of claim 17, the polymer layer comprising polyvinylpyrrolidone.

20. The medical device of claim 17, the polymer layer comprising an elution control matrix.
